(19) United States
(12) Patent Application Publication
(10) Pub. No.: US 2019/0320979 A1
(43) Pub. Date: Oct. 24, 2019

(54) METHOD AND SYSTEM FOR COLLECTING AND PROCESSING BIOELECTRICAL SIGNALS

(71) Applicant: Emotiv Inc., San Francisco, CA (US)

(72) Inventors: Tan Le, San Francisco, CA (US); Geoffrey Ross Mackellar, Sydney (AU); William King, Manly (AU); Nam Nguyen, North Sydney (AU)

(21) Appl. No.: 16/391,071

(22) Filed: Apr. 22, 2019

Related U.S. Application Data
(60) Provisional application No. 62/660,842, filed on Apr. 20, 2018.

(51) Int. Cl. A61B 5/00; A61B 5/0476; G06K 9/62; A61B 5/04

(52) U.S. Cl. CPC A61B 5/684; A61B 5/0476; A61B 5/7221; A61B 5/6843; A61B 5/6803; G06K 9/6263; A61B 5/7207; A61B 5/04014; A61B 5/743; A61B 5/7267

(57) ABSTRACT

A variation of a method for collecting and processing bioelectrical signals includes: establishing bioelectrical contact between a user and one or more sensors of a biomonitoring neuroheadset; monitoring contact characteristics of the one or more sensors based on bioelectrical signals detected at the one or more sensors; and providing feedback to the user based on the contact characteristics. A variation of a system for collecting and processing bioelectrical signals includes a set of sensors (e.g., electrodes) and a processing subsystem configured process the set of bioelectrical signals.

### CROSS-REFERENCE TO RELATED APPLICATIONS

This application claims the benefit of U.S. Provisional Application Ser. No. 62/660,842 filed 20 Apr. 2018, which is incorporated in its entirety herein by this reference.

### TECHNICAL FIELD

This invention relates generally to the field of digital signal collection and processing, and more specifically to a new and useful method and system for collecting, processing, and analyzing bioelectrical signals.

### BACKGROUND

In order to record high quality EEG data, it is important to establish good electrical contact with the user (e.g., at the scalp). In many EEG recording systems, the setup process includes using sandpaper to remove dead skin cells, and then mediating the electrical contact by the use of a conductive gel into which the electrodes are placed. An experienced technician may then be able to verify that there is a good electrical contact by visual inspection of the EEG signals or other methods. However, in some settings (e.g., consumer settings, non-clinical settings, etc.), the user (e.g., consumer) may not have the requisite experience and/or knowledge to determine that satisfactory electrical contact has been obtained, nor access to an experienced technician. Furthermore, the use of a conductive gel may not be appropriate or desirable in many settings (e.g., consumer settings, athletic settings, humid settings, marine settings, social settings, etc.). Thus, there is a need in the field of bioelectrical signal analysis for a new and useful system and/or method for establishing and maintaining electrical contact between a bioelectrical monitoring system and a user.

### BRIEF DESCRIPTION OF THE FIGURES

FIG. 1 depicts a flowchart of an embodiment of a method for bioelectrical contact quality monitoring;

FIG. 2 depicts a schematic illustration of an embodiment of a method for bioelectrical contact quality monitoring;

FIG. 3 depicts a schematic illustration of a portion of an embodiment of a method for bioelectrical contact quality monitoring;

FIG. 4 depicts a specific example of a portion of the method for bioelectrical contact quality monitoring including noise artifact detection and mitigation;

FIG. 5 depicts a schematic illustration of an embodiment of a system for bioelectrical contact quality monitoring;

FIG. 6 depicts a schematic illustration of an embodiment of a system and method for bioelectrical contact quality monitoring;

FIG. 7 depicts a schematic illustration of an embodiment of a method; and

FIG. 8 depicts a schematic illustration of feedback indicating sensor contact quality.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Overview

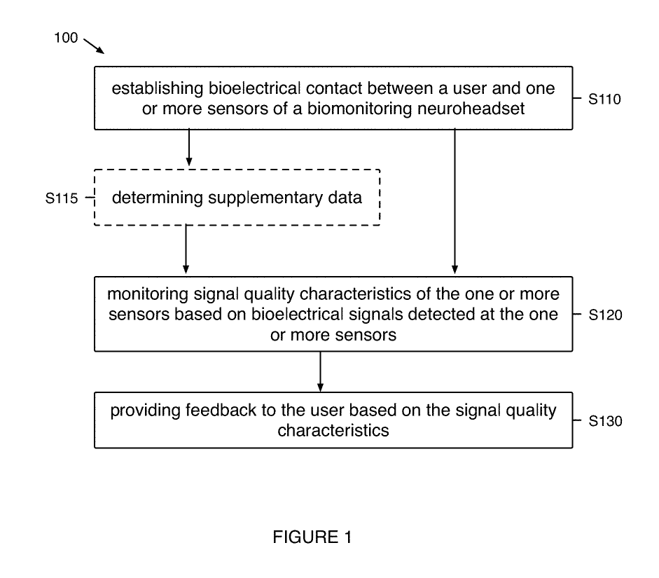

As shown in FIG. 1, an embodiment of a method 100 for collecting and processing bioelectrical signals includes: establishing bioelectrical contact between a user and one or more sensors of a biomonitoring neuroheadset S110; monitoring contact characteristics of the one or more sensors based on bioelectrical signals detected at the one or more sensors S120; and providing feedback to the user based on the contact characteristics S130. The method 100 functions to ensure optimal electrical contact between the one or more sensors and the user (e.g., at a head region of the user, at an ear region of the user, etc.) such that bioelectrical signals can be accurately and efficiently obtained (e.g., measured in the presence of excess noise, measured in the absence of excess noise, obtained with an adequate signal-to-noise ratio, etc.). The method 100 can also function to provide feedback to the user to enable the user to self-adjust the positioning and/or other characteristics of the sensors in order to maintain and/or improve bioelectrical contact. The method 100 can also function to determine the presence of artifacts in the bioelectrical signals that are indicative of problematic aspects (e.g., lack of stability, lack of sensitivity, etc.) of the established bioelectrical contact, and to provide notification(s) related to the determined artifacts to the user.

The method 100 can additionally or alternatively include: determining supplementary data S115 (e.g., usable as a basis for monitoring contact characteristics). Supplementary data can, in variations, include contextual data (e.g., data collected contemporaneously with bioelectrical signal data, data collected that is related to bioelectrical signal data but collected at a different time and/or retrieved from a database, data collected from a motion sensor, etc.). However, the method 100 can additionally or alternatively include any other suitable techniques for monitoring and maintaining high quality bioelectrical contact between one or more bioelectrical sensors and a user.

In relation to the method 100, signal features include aspects of the signals (e.g., bioelectrical signals, EEG signals, supplementary signals, etc.) that are derived, extracted, or otherwise suitably determined from the raw data. For example, signal features can include any one or more of: frequency content (e.g., a frequency domain transform of a time domain signal, power as a function of frequency across a plurality of frequency bands, etc.), peak characteristics (e.g., number of peaks, width of peaks, amplitude of peaks, etc.), time-domain content (e.g., time-series dynamics, signal shapes, signal power as a function of time, etc.), and any other suitable features of the signals. Signal features can, in variations, be indicative of properties of the user (e.g., a time-dependent bioparameter, cognitive state, basal bioelectrical output, movements, head gestures, etc.), properties of the biomonitoring device (e.g., contact quality, physical orientation, positional stability in relation to the user, power levels, etc.), and any other suitable user and/or device characteristics.

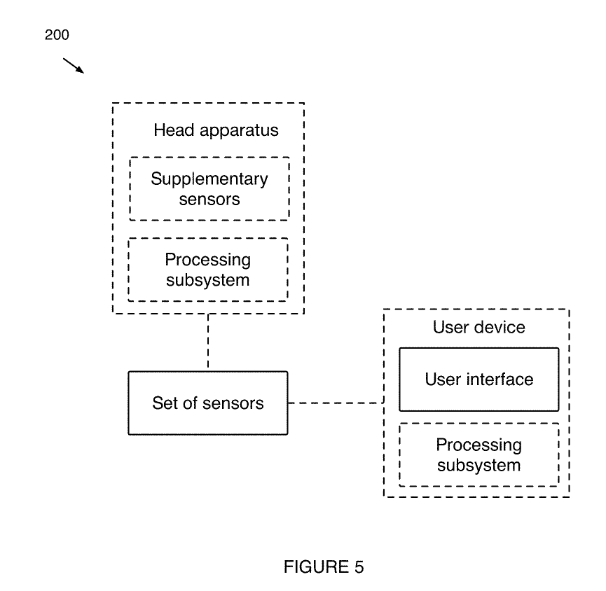

The method 100 is preferably implemented, executed, or otherwise performed at and/or in conjunction with a system 200 (e.g., as shown in FIG. 5). The system 200 preferably includes a biomonitoring headset substantially as described in U.S. application Ser. No. 15/209,582, entitled “Method and System for Collecting and Processing Bioelectrical and Audio Signals” and filed 13 Jul. 2016, which is incorporated herein in its entirety by this reference. The system 200 can additionally or alternatively include a remote computing system (e.g., remote from the user, a remote server, a cloud-based computing system, etc.), a mobile device of the user (e.g., a smartphone, a laptop, a tablet, etc.), one or more networked sensors (e.g., a networked thermostat, an internet-connected video camera, etc.), and any other suitable computing resources or suitable components.

As shown in FIG. 5, an embodiment of a system 200 for collecting and processing bioelectrical signals includes a set of sensors (e.g., electrodes) configured to receive a set of bioelectrical signals (e.g., EEG signals) from a user and a processing subsystem configured process the set of bioelectrical signals. Additionally or alternatively, the system 200 can include any or all of: a user interface (e.g., display, speaker, etc.) configured to provide a notification to the user, a head apparatus (e.g., biomonitoring headset, headphones, headband, earbuds, etc.), a user device, any number of supplementary sensors (e.g., to record supplementary data), and any other suitable component.

### 2. Benefits

Variants of the method for collecting and processing bioelectrical signals can afford several benefits and/or advantages.

First, variants of the system and method enable a user that lacks experience and/or knowledge in recognition of EEG signal quality to optimize neuroheadset performance using simple, actionable feedback provided to the user, which prompts and/or permits the user to adjust sensors to obtain proper contact if needed, and which provides feedback indicating adequate contact when no further action is required. The feedback preferably includes automatically-generated user feedback, but can additionally or alternatively include any suitable feedback. In some examples of the method, for instance, visual graphics are provided to the user (e.g., through the display of a user device) which indicate which electrodes require adjustment.

Second, variants of the system and method can confer benefits over conventional manual (e.g., visual) artifact detection and/or analysis. For example, variants of the method can automatically extract signal features (e.g., for each of a set of signal frequency bands) from each instance of a sliding time window, and automatically feed the signal features and/or derivative data (e.g., signal RMS, sum of the absolute gradient at each point, etc.) into a model (e.g., a Gaussian model), wherein the model can automatically determine a contact quality score in real or near-real time. This can both improve the user experience for an inexperienced user and improve the way the system (e.g., a computing system) stores data, retrieves data, and determines electrode contact. Further examples can dynamically adjust the signal detection sensitivity, which can balance user adoption of the headset with collecting high-quality signals. In a specific example, analysis of signal data (e.g., in order to extract bioparameter of a user, in order to extract mental states of a user, etc.) is enhanced by labeling one or more signal data streams (e.g., signal stream from a single electrode, aggregated signal stream from multiple electrodes, etc.) with a quality metric. The quality metric can be used to weight the data, exclude or apply selective intensive artifact removal techniques (e.g., independent components analysis) to selected time windows depending upon one or more instantaneous signal quality metrics, or can be used in any other suitable way.

Third, variants of the system and method can include investigating multiple different features contributing to signal quality. When used together, these can function, for instance, to enable a robust and comprehensive assessment of signal quality (e.g., with greater than 80% confidence, greater than 90% confidence, etc.). Additionally or alternatively, these multiple different features can provide greater insight into the source and/or solution to a signal quality issue. In an example, for instance, a slipping electrode can be distinguished from noise within the system.

However, variants of the method for collecting and processing bioelectrical signals can additionally or alternatively afford any suitable benefits and/or advantages.

### 3. Method

As shown in FIG. 1, an embodiment of a method 100 for collecting and processing bioelectrical signals includes: establishing bioelectrical contact between a user and one or more sensors of a biomonitoring neuroheadset S110; monitoring contact characteristics of the one or more sensors based on bioelectrical signals detected at the one or more sensors S120; and providing feedback to the user based on the contact characteristics S130.

The method 100 can additionally or alternatively include: determining supplementary data S115 (e.g., usable as a basis for monitoring contact characteristics). Supplementary data can, in variations, include contextual data (e.g., data collected contemporaneously with bioelectrical signal data, data collected that is related to bioelectrical signal data but collected at a different time and/or retrieved from a database, data collected from a motion sensor, etc.). However, the method 100 can additionally or alternatively include any other suitable techniques for monitoring and maintaining high quality bioelectrical contact between one or more bioelectrical sensors and a user.

### 3.1 Method: Establishing Bioelectrical Contact S110

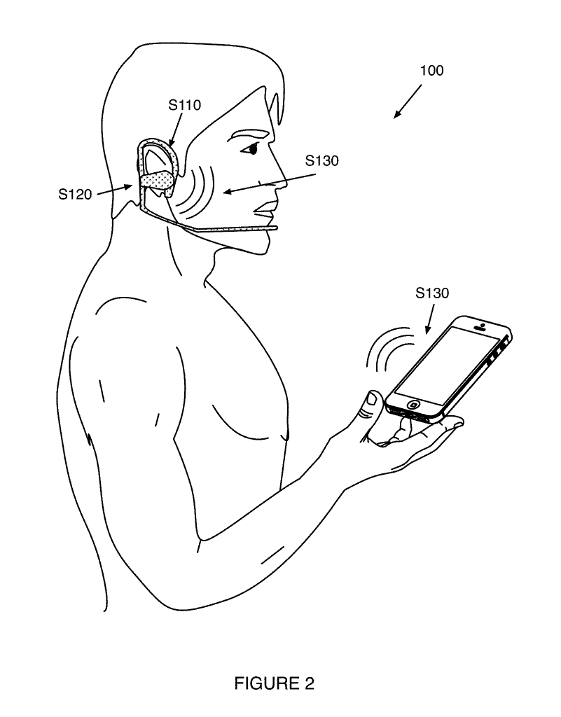

As shown in FIG. 2, Block S110 recites: establishing bioelectrical contact between a user and one or more sensors of a biomonitoring neuroheadset, which functions to facilitate a bioelectrical interface between an individual and a biosignal detector. Establishing bioelectrical contact S110 is preferably between one or more sensors of a biomonitoring neuroheadset and a human, but can additionally or alternatively be with a biomonitoring neuroheadset and any other suitable organism (e.g., a pet, an animal, etc.). One or more bioelectrical sensors of the biomonitoring neuroheadset preferably include one or more EEG sensors and one or more reference sensors (e.g., common mode sensor, sensors associated with a driven right leg module, etc.). Alternatively, the biomonitoring neuroheadset can omit reference sensors. However, the biomonitoring neuroheadset can additionally or alternatively include any bioelectrical signal sensors configured to detect any one or more of: electrooculography (EOG) signals, electromyography (EMG) signals, electrocardiography (ECG) signals, galvanic skin response (GSR) signals, magnetoencephalogram (MEG) signals, and/or any other suitable signal.

Relating to Block S110, bioelectrical contact is preferably established through sensors arranged at a particular location or region of the user (e.g., head region, torso region, etc.). For example, Block S110 can include establishing bioelectrical contact between a first subregion of an ear region of the user and an EEG sensor of a biomonitoring neuroheadset. In a specific example, the first subregion of the ear region (e.g., an ear region of a left ear) can include an ear canal (e.g., a left ear canal) of the user. In a variation of Block S110 where the biomonitoring neuroheadset includes a set of EEG sensors, Block S110 can include establishing bioelectrical contact between a first contralateral subregion of a contralateral ear region (e.g., an ear region of a right ear) of the user and a second EEG sensor of the biomonitoring neuroheadset, where the first contralateral subregion can include a contralateral ear canal (e.g., a right ear canal) of the user.

In another variation of Block S110 where the biomonitoring neuroheadset includes one or more common mode sensors Block S110 can additionally or alternatively include establishing bioelectrical contact between a second subregion of the ear region of the user and a common mode sensor of a noise reduction subsystem of the biomonitoring neuroheadset S112. In a specific example of the variation, the second ear subregion is proximal the first subregion, and the EEG sensor is proximal the common mode sensor. In another specific example, Block S110 can include establishing bioelectrical contact between a second contralateral subregion of the contralateral ear region of the user and a second common mode sensor of a noise reduction subsystem of the biomonitoring neuroheadset, where the first contralateral subregion is proximal the second contralateral subregion, and where the second EEG sensor is proximal the second common mode sensor. In this specific example, the second subregion can include an ear subregion proximal a mastoid process of a temporal bone of the user, and where the second contralateral subregion can include a contralateral ear subregion proximal a contralateral mastoid process of a contralateral temporal bone of the user.

In another variation of Block S110 where the biomonitoring neuroheadset includes one or more driven right leg (DRL) sensors of a driven right leg module, Block S110 can include establishing bioelectrical contact between a third subregion of the ear region of the user and a DRL sensor of a DRL module of the noise reduction subsystem. The third subregion is preferably at an ear region (e.g., proximal a mastoid process of a temporal bone of the user), but can alternatively be at any suitable anatomical position of the user.

In variations of Block S110, establishing bioelectrical contact can include any elements analogous to those disclosed in U.S. patent application Ser. No. 13/903,861 filed 28 May 2013, U.S. patent application Ser. No. 14/447,326 filed 30 Jul. 2014, and U.S. patent application Ser. No. 15/058,622 filed 2 Mar. 2016, which are hereby incorporated in their entirety by this reference. However, establishing bioelectrical contact between body regions of a user and different types of sensors can be performed in any suitable manner.

### 3.2 Method: Determining Contextual Data S115

The method 100 can include Block S115, which includes determining contextual data. Contextual data is preferably any data that is distinct from the bioelectrical signals and can be used to aid determination of contact quality in subsequent Blocks (e.g., Block S120) of the method 100. In a first example, Block S115 can include determining a geographic location of the user (e.g., from a manual location entry, a GPS signal from the headset or connected user device, etc.), and thereby determining the expected frequency of mains noise and/or artifact patterns associated with the geographic location of the user (e.g., 50 Hz in countries wherein electrical mains noise is present primarily at 50 Hz and harmonics thereof, 60 Hz in countries wherein electrical mains noise is present primarily at 60 Hz and harmonics thereof, etc.). In a second example, Block S115 includes detecting an audio signal, and identifying features in the audio signal that can be used to generate a comparison in accordance with subsequent Blocks of the method 100 (e.g., Block S120) between extracted artifacts and the features of the audio signal (e.g., to eliminate bioelectrical contact quality as a cause of the extracted artifacts). In a third example, Block S115 includes detecting movements of the user (e.g., head gestures, steps, sharp movements, collisions or other movements which may temporarily or permanently dislodge, slide, or otherwise compromise biosignals, etc.) and identifying features of the motion signals which can be used to generate a comparison in accordance with subsequent processes of the method. However, Block S115 can include determining any suitable type of contextual data in any suitable manner.

### 3.3 Method: Monitoring Signal Quality Characteristics S120

As shown in FIG. 1, the method 100 includes Block S120, which includes: monitoring signal quality characteristics of the one or more sensors based on bioelectrical signals detected at the one or more sensors. Block S120 functions to facilitate collection of high quality sensor signals (e.g., bioelectrical signals) through proper (e.g., adequate, ideal, purposeful, etc.) coupling between the user and one or more sensors of the biomonitoring device (e.g., neuroheadset). Signal quality characteristics can include contact characteristics (e.g., contact quality, contact stability, etc.), other signal characteristics (e.g., noise, signal-to-noise ratio [SNR], energy, signal artifacts, etc.), and any other suitable aspects of bioelectrical contact and the resulting signals transmitted between the one or more sensors and the user.

Signal quality characteristics (e.g., contact characteristics) are preferably monitored for one or more bioelectrical signal sensors (e.g., contact quality between an EEG sensor and an ear canal region, contact stability for a collection of EEG sensors positioned at a head region of the user, etc.) and/or reference signal sensors (e.g., contact quality between a common mode sensor and an ear region proximal the temporal bone; contact quality between a driven-right-leg sensor and an ear region proximal the temporal bone, etc.). However, monitoring signal quality characteristics can be performed for any suitable sensor. Monitoring signal quality characteristics is preferably performed for a sensor with a target position proximal an ear region of a user, but can additionally or alternatively be performed for sensors with target positions at any suitable anatomical position of a user. However, monitoring signal quality can be performed for any suitable sensor at any suitable location.

With respect to temporal aspects relating to Block S120, signal quality is preferably continuously monitored, in order to facilitate immediate real-time feedback to a user in response to detection of an uncoupling state and/or poorly-coupling state, a signal artifact, or any other signal quality issue between one or more sensors and the user. Block S120 can additionally or alternatively be performed at any suitable frequency in a periodic manner (e.g., every 0.25 seconds, every 0.5 seconds, every 10 seconds, etc.). Monitoring signal quality S120 can additionally or alternatively be associated with and/or performed during a temporal indicator (e.g., a time period prior to collection of a bioelectrical signal dataset, in response to a detected trigger, etc.), but can otherwise be performed at any suitable time in relation to other portions of the method 100.

In a variation, Block S120 can include applying a reference signal with one or more sensors. In this variation, a reference signal preferably characterized by low voltage and low current can be applied to the user by one or more sensors (e.g., one or more electrodes of the biomonitoring neuroheadset). The reference signal can be a square wave, a sine wave, another suitable waveform, an impedance measure, and/or any other applicable reference signal. However, the reference signal can possess any suitable properties.

In relation to this variation of Block S120, one or more reference signals are preferably applied by one or more reference signal sensors (e.g., a common mode sensor, a DRL sensor of a DRL module, etc.), but can otherwise be applied by any suitable sensor. This can function to provide a direct electrical measurement of the headset contact with the user. In examples where a DRL sensor applies a reference signal, the reference signal can be combined with a biasing signal and injected through a DRL electrode positioned at a feedback reference point of the DRL module. In a specific example, the DRL module applies a square wave potential (e.g., a square wave potential is injected into the DRL signal) and the output potential is measured at each sensor (e.g., electrode) of the bioelectrical monitoring device, and the properties of the transfer function between the injected signal and the detected signal are determined via analysis of the measured output potential (e.g., by computing the transfer function that transforms a square wave into the measured signal, by phase locked detection of the transmitted square wave component detected at each biosensor location, etc.). However, sensors applying reference signals can be characterized by any suitable trait.

In some variations, a direct electrical measurement process, such as that described above, can serve as a trigger (e.g., first check in a signal quality detection workflow) for subsequent signal quality detection processes. In a specific example, for instance, a direct electrical measurement is taken to predict or determine whether or not the headset is in proper contact with the head of the user. In the event that the electrical measurement indicates proper contact (yet the signal quality is below a threshold), further analysis can be initiated. In the event that the electrical measurement indicates improper contact, a notification to the user can be provided indicating that the user should adjust one or more electrodes (e.g., remove the headset and place in a new position). In another example, further analysis is always initiated as, in some cases, a false positive of proper contact can occur. In some cases, for instance, it can be suggested from direct electrical measurement alone that an electrode headset, that is completely disconnected from a user (e.g., sitting on a table top), is actually in proper contact with the head of a user, as square wave signals sent to the set of electrodes during the electrical measurement process can elicit a harmonic response similar to that corresponding to proper contact.

Alternatively, the method 100 can be performed in absence of the direct electrical measurement process, the direct electrical measurement process can be performed contemporaneously with (e.g., during) or after other signal quality detection processes, the direct electrical measurement process can initiate any other suitable triggers, or the method 100 can be performed in any other suitable way.

In some variations of Block S120, monitoring signal quality can include generating a signal quality metric. Generated signal quality metrics preferably indicate a quality of the signal content (e.g., taking into account the amount of noise in the signal, taking into account the type of noise in the signal, taking into account a signal-to-noise ratio, taking into account a signal artifact, other signal parameter, etc.), but can additionally or alternatively include one or more contact quality metrics, (eg. impedance measurement, etc.) which preferably indicate the quality of coupling between a sensor and a user for accurately collecting biosignals. Generated signal quality metrics can possess any suitable form, including numerical (e.g., probabilities of sufficient contact quality, raw values, processed values, etc.), verbal (e.g., verbal indications of contact quality, etc.), graphical (e.g., colors indicating level of contact quality, educational graphics for facilitating improved contact quality, etc.), and/or any suitable form. Generating a signal quality metric is preferably performed at a remote processing module (e.g., cloud computing system, remote server, processing module of a user device, etc.) communicatively coupled (e.g., wirelessly connected) to the biomonitoring neuroheadset, but can additionally or alternatively be performed at a processing module of the biomonitoring headset, at any suitable user device, any suitable remote server, and/or any suitable component. In one variation, a contact quality metric is measured using a square wave system within a processing module of the biomonitoring headset, while subsequent signal quality metrics are calculated in a separate processing module (e.g., at a receiving computing device, mobile device, personal computer, tablet, etc.). Alternatively, multiple metrics can be determined at a single processing module (e.g., at the biomonitoring headset, at a user device, at a remote server, etc.), at another set of multiple processing modules, or at any suitable number and arrangement of processing modules. However, generating a signal quality metric can be otherwise performed.

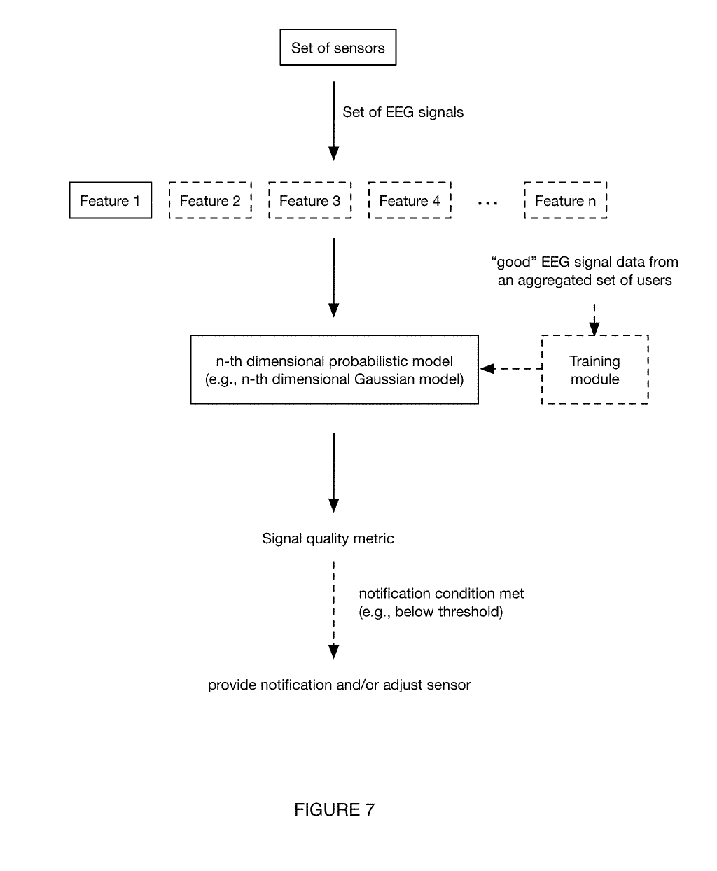

Block S120 can include implementing a signal quality model. The signal quality model functions to assess the signal content of one or more signal streams, preferably in relation to noise and/or any potential artifacts within the signal. The signal quality model can additionally or alternatively include a contact quality model, which functions to assess the contact of one or more sensors (e.g., based on real-time impedance measurements) with the user. Implementing the signal quality model preferably includes comparing monitored bioelectrical signals with predicted signal features that are predicted by the signal quality model. The comparison preferably results in an output (e.g., signal quality metric) that includes the covariance between extracted features of the bioelectrical signals and the predicted features of the model (e.g., the likelihood and/or log-likelihood that the extracted features were sampled from the expected distribution corresponding to high quality contact data), but can additionally or alternatively result in any suitable form of comparison (e.g., phase, magnitude, frequency, etc.). The signal quality model is preferably a multi-dimensional Gaussian model (e.g., as shown in FIG. 7) wherein a Gaussian function describes each feature of the signal, but can additionally or alternatively be any suitable probabilistic model, a classification model (e.g., wherein signal patterns or derivatory data patterns can be associated with an artifact class), neural network, or any other suitable model.

The signal quality model is preferably determined based on data aggregated from multiple users. Additionally or alternatively, one or more signal quality models can be determined based on data from a single user (e.g., to determine a model specific to a particular user), synthetic data, or any other suitable data. The signal quality model is preferably dynamic and routinely updated (e.g., with the introduction of additional data, on an annual basis, etc.), but can additionally or alternatively be otherwise updated (e.g., in response to a trigger such as a malfunction, as determined by a user, etc.), static, or any combination of static and dynamically updated.

The signal quality model is further preferably determined based on data having a signal quality above a predetermined threshold (“good” data), as determined for instance, by an expert or professional trained in proper EEG electrode placement (e.g., EEG technician, physician, neuroscientist, etc.). Developing the model based on only “good” data can be beneficial. In some cases, for instance, the amount of data required to develop the model can be relatively minimal, as capturing and recording data from each of the many scenarios causing poor signal quality are not required. As the scenarios which result in poor signal quality are not only numerous but also potentially rare, complex, and difficult to capture, this can be particularly advantageous. Additionally or alternatively, however, one or more models corresponding to and/or classifying data of poor signal quality (e.g., positively identifying an electrode being tapped on by the finger of a user) can be determined.

The set of signal features determined from the monitored bioelectrical signals and compared with the signal quality model can include any suitable number and type of features. Preferably, multiple categories of features are determined, which can function to produce any or all of: an increased robustness of the method, a more specific determination of the cause of poor contact (e.g., electrode sliding versus poor electrode placement), a more specific determination of a solution to correct for poor contact, or any other suitable outcome. In one variation, each of the multiple categories of features enables a different targeted solution for improving signal quality to be suggested to the user. In a second variation, a proposed solution is not associated with any particular signal feature.

Block S120 can optionally include a windowing process, wherein each window of signal data is analyzed to determine a set of signal features, which are then compared with the signal quality model. The windowing process is further preferably a sliding window process; additionally or alternatively, Block S120 can include a tumbling window process, or any other suitable windowing process. Each of the windows is preferably less than 10 seconds, further preferably between 1 and 3 seconds (e.g., 2 seconds), but can additionally or alternatively be greater than 10 seconds (e.g., 30 seconds, between 0 and 30 seconds, 1 minute, 2 minutes, between 0 seconds and 2 minutes, less than 10 minutes, greater than 10 minutes, etc.), between 0 and 1 second, variable, or otherwise determined. In variations including a sliding window process, the set of features are preferably updated more frequently than every second (e.g., every eighth second, every quarter second, every half second, etc.), but can alternatively be updated less frequently than every second.

In one variation, Block S120 analyzes signal data through a sliding window process, wherein each window is 2 seconds long, and wherein the set of signal features are updated every quarter second.

The set of signal features preferably includes a parameter (e.g., power, frequency, phase, etc.) associated with a predetermined frequency range (frequency band), further preferably a parameter associated with each of a set of frequency ranges. In variations having multiple frequency ranges, the frequency ranges can be overlapping, non-overlapping, spaced apart, or otherwise selected. The parameter associated with a frequency range is herein equivalently referred to as a frequency feature. In preferred variations, the parameter is a power, but can additionally or alternatively include any suitable parameter, such as—but not limited to—a frequency, phase, time, or amplitude. For multiple frequency ranges, the parameter type is preferably the same for each frequency range (e.g., power for each frequency range), but additionally or alternatively: the parameter can be of a different type for different frequency ranges, multiple parameters can be determined for each frequency range, or any other suitable parameters associated with any number of frequency ranges can be determined.

The frequency features can function to identify one or more artifacts present in the signal, especially artifacts manifesting at particular frequencies or ranges of frequencies. Noise from a mains frequency (equivalently referred to as a utility frequency), for instance, is typically associated with a particular or range of frequencies (e.g., based on the country where the user is located). One or more frequency features can correspond to the mains frequency or frequencies (e.g., between 50 and 60 Hz) to thereby detect when a signal artifact can be attributed to mains noise. Signal artifacts associated with a frequency outside of this range may indicate a less common source of poor signal quality and therefore trigger the suggestion of solution for establishing better, more stable electrode contact with the user. Frequency ranges corresponding to other artifacts can additionally or alternatively be included.

In some variations, the contamination of signals by mains frequency signals are often accompanied by higher harmonics of the mains frequency (e.g., 100 Hz, 150 Hz, 450 Hz for a mains frequency of 50 Hz, etc.) and if sufficiently dominant, these frequencies can overcome the normal filtering designed to remove them prior to sampling. In this event, these high frequency signals may appear as low frequency components within a biosignal (e.g., EEG) frequency band of interest through a sample aliasing process (e.g., 120 Hz harmonic content sampled at 128 Hz will produce an artifact signal at 8 Hz—the absolute difference between the harmonic content frequency and a multiple of the sampling frequency—along with alias signals). In a specific example, the method targets this artifact frequency component. Additionally or alternatively, the method can target any other artifact frequency components.

The frequency ranges can additionally or alternatively include part or all of physiological frequency bands (e.g., brain waves, alpha band, beta band, delta band, theta band, etc.). In one variation, if a power associated with a particular physiological frequency band (e.g., alpha band) is outside of predetermined physiological range, it can be determined that any signal artifacts in this range are associated with an electrode contact issue, another component of the device (e.g., motor), or any other suitable source.

In one variation, the set of signal features includes the power associated with each of a set of multiple frequency bands, the set of multiple frequency bands approximately centered around a mains frequency (e.g., average mains frequency, mains frequency for the particular country of the user, etc.) of the user. In a specific example, powers associated with the following frequency bands are determined: 41-48.5 Hertz (Hz), 49-51 Hz, 51.5-58.5 Hz, 59-61 Hz, and 61.5-64 Hz.

The set of signal features can additionally or alternatively include an energy (e.g., root mean square [RMS] power) associated with the signal received at one or more electrodes. This can function to identify scenarios which may deviate from a norm, such as those in which a high energy signal is associated with low level of noise, and those in which a low energy signal is associated with a high level of noise.

In the variation described previously in which a set of electrodes not in contact with a user produces a signal based on the harmonics of the device, the energy of the signal (e.g., low energy, energy below a physiological threshold, etc.) can properly indicate that the device is not in contact with the user.

The set of signal features can further additionally or alternatively include a gradient parameter (e.g., absolute gradient at a time point, sum of the absolute gradient at each time point, overall absolute gradient for a set of multiple time points, maximum gradient value for each of a predetermined set of time points, gradient within the frequency spectrum measured at specific frequencies at a point in time, etc.) associated with the signal received at one or more electrodes. This can function to indicate how much the signal is changing over time. A spike in the signal, for instance, can indicate a sudden change in electrode contact (e.g., electrode movement, electrode falling off the user, etc.).

In one variation of the set of signal features, the set of signal features includes the sum of the absolute gradient of the signal at each of a set of time points (e.g., as compared with a previous window of signal data, as compared with a predicted signal, etc.).

In a second variation of the set of signal features, the set of signal features includes a gradient measure in frequency space which would identify spikes from mains noise and aliased harmonics (e.g., which tend to be sharp spikes).

In one variation of the signal quality model, the signal quality model includes a multi-dimensional probabilistic model (e.g., a Gaussian model) that functions as a reference model (e.g., is trained on “good” or “clean” data). In this example, each dimension of the probabilistic model corresponds to a different feature extracted from the signal, wherein the model can represent the typical covariance between the different features. In operation, signal features can be extracted from the signals and compared against the model. In a first specific example, comparing the extracted features against the model includes determining a probability that the extracted values were sampled from the probabilistic model and/or fit within the probabilistic model, wherein the determined probability can function as the signal quality metric. In this example, the signal can be classified as “good” when the probability is high (e.g., above a predetermined threshold), and classified as “bad” when the probability is low (e.g., below the same or a different predetermined threshold). In a second specific example, comparing the extracted features against the model includes outputting a probability of the signals or contact being “good,” wherein the signals or contact can be classified as “bad” when the probability falls below a predetermined threshold. However, the signal quality metric can be otherwise determined. In this example, the sensitivity of the detection can optionally be adjusted based on: the desired signal quality, the ease of use, the amount of time required for a user to establish good contact (e.g., contact with a signal quality metric above a predetermined threshold), and/or any other suitable variable. The variable value can be determined based on: use history, the use case (e.g., application), or otherwise determined.

Block S120 can optionally include automatically adjusting the biomonitoring neuroheadset to establish bioelectrical contact between one or more sensors and the user in response to monitoring the bioelectrical signals. Automatically adjusting the biomonitoring neuroheadset can include: directing the orientation of one or more sensors of the biomonitoring neuroheadset (e.g., automatically orienting one or more sensors towards a target anatomical position of the user), providing an actuating force (e.g., a vibration, a biasing force, a pulsating force, etc.) to move one or more sensors into bioelectrical contact with the user, adjusting data collection parameters of one or more sensors (e.g., a bias voltage, a feedback current magnitude, a feedback voltage magnitude, a collection frequency, a duty cycle, etc.), releasing contact fluid or gel from a reservoir (e.g., within a biomonitoring headset, attached to a biomonitoring headset, etc.), and/or any other suitable adjustments of the biomonitoring neuroheadset. Automatically adjusting the biomonitoring neuroheadset is preferably performed in response to detecting an unsuitable contact quality and/or any other signal quality during collection of biosignals, but can additionally or alternatively be performed at any suitable time and/or with any suitable temporal characteristics. However, automatically adjusting the biomonitoring neuroheadset can be performed in any suitable manner.

In relation to Block S120, artifacts detected in the bioelectrical signals gathered in accordance with monitoring bioelectrical contact quality can include artifacts generated due to: electrical power mains (e.g., mains noise), low frequency drift, voltage steps, voltage jumps, and any other relevant signal artifacts.

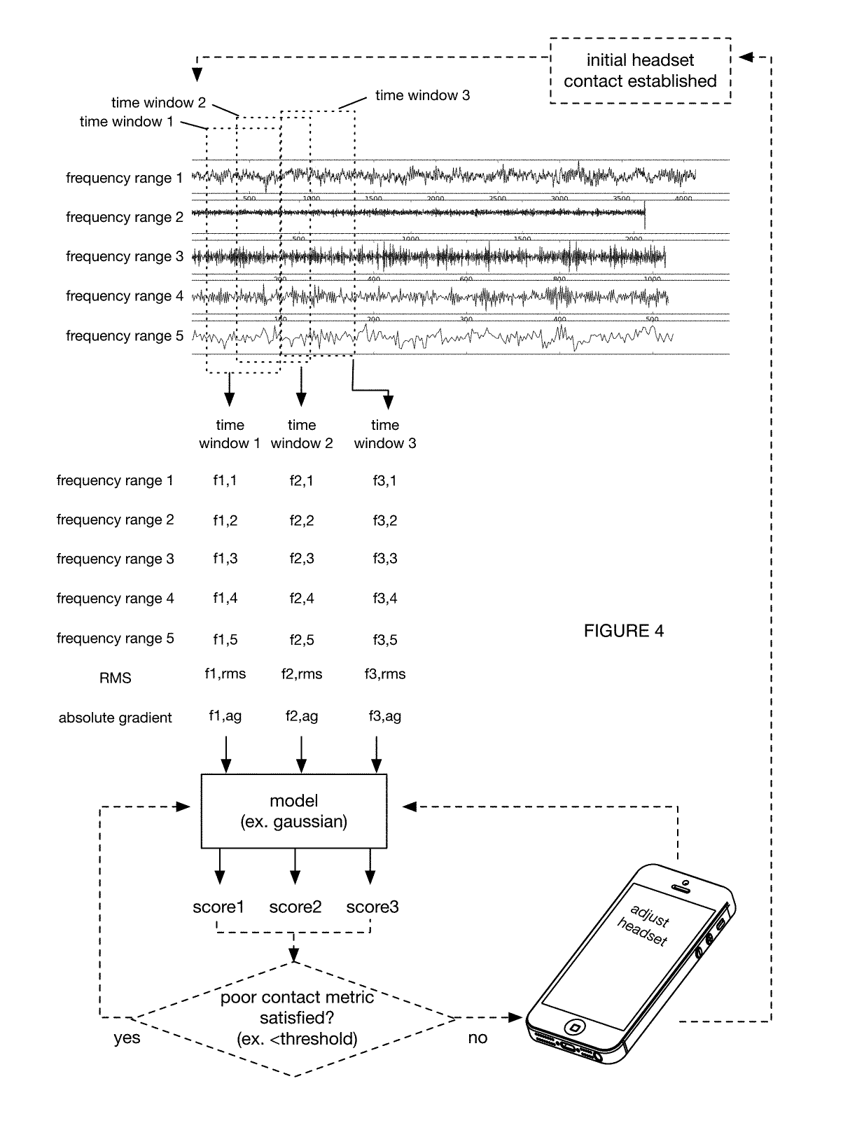

In one variation (e.g., as shown in FIG. 4), the signal quality model is a seven-dimensional Gaussian model, wherein five of the seven dimensions correspond to a band power in each of five frequency bands including: 41-48.5 Hz, 49-51 Hz, 51.5-58.5 Hz, 59-61 Hz, and 61.5-64 Hz. The remaining two of the seven dimensions of this specific example correspond to the RMS value of each signal and the sum of the absolute gradient at each time point, respectively, of the five bioelectrical signals (e.g., from each of the five bands). Each of these seven features in this example are processed in sliding averaging windows (e.g., two seconds in duration, one second in duration, etc.). The signal quality model preferably outputs a signal quality metric (e.g., as described above), but can additionally or alternatively provide any suitable output. The output of the signal quality model is preferably provided in substantially real-time, but can additionally or alternatively be provided with any suitable temporal characteristics (e.g., asynchronously, logged for future analysis, etc.). The signal quality model can be generated (e.g., trained, validated, updated, etc.) based on a supervised training data set (e.g., generated by an expert applying the biomonitoring neuroheadset to a user), a simulated training data set, a historic data set (e.g., for the user, for a user population, filtered for manually or otherwise determined high-quality signals, etc.), or based on any other suitable data.

In a second variation, a signal quality model is determined, selected, or adjusted based on any or all of the following: a number of signal channels (e.g., 2-channel EEG, 16-channel EEG, 14-channel EEG, 5-channel EEG, between 1- and 20-channel EEG, greater than 20-channel EEG, etc.), user information (e.g., user demographics, age, gender, ethnicity, etc.), environmental information (e.g., as determined based on collected supplementary data, environmental noise, motion, etc.), or any other suitable information.

Additionally or alternatively, Block S120 can include any elements and/or techniques substantially as described in U.S. patent application Ser. No. 12/270,739, filed 13 Nov. 2008, which is herein incorporated in its entirety by this reference. However, Block S120 can be otherwise performed in any suitable manner.

### 3.4 Method: Providing Feedback to the User S130

As shown in FIG. 1, the method includes Block S130, which includes: providing feedback to the user based on the contact characteristics. Block S130 functions to enable the user of the biomonitoring device to improve the performance of bioelectrical signal collection in real- or near-real-time without requiring the user to have specialized knowledge or skill in bioelectrical signal monitoring. Block S130 can also function to inform the user that contact is suboptimal (e.g., without providing further instructions on specific sensor placement changes) and encouraging the user to adjust the biomonitoring device. In such cases, specific guidance may not be necessary and the user can iteratively adjust the placement of the biomonitoring device and be automatically informed that the bioelectrical contact is or is not sufficient for bioelectrical signal collection (e.g., until the contact quality metric satisfies a predetermined value, until the direct electrical measurement satisfies a predetermined condition, etc.). Additionally or alternatively, specific guidance (e.g., electrode-specific instructions, an ordered list of adjustments for the user to make, instructions to adjust the biomonitoring device in a particular direction or set of directions, instructions to rotate the biomonitoring device about a particular axis, instructions to adjust a relative spacing between two or more of a set of electrodes, etc.) can be provided.

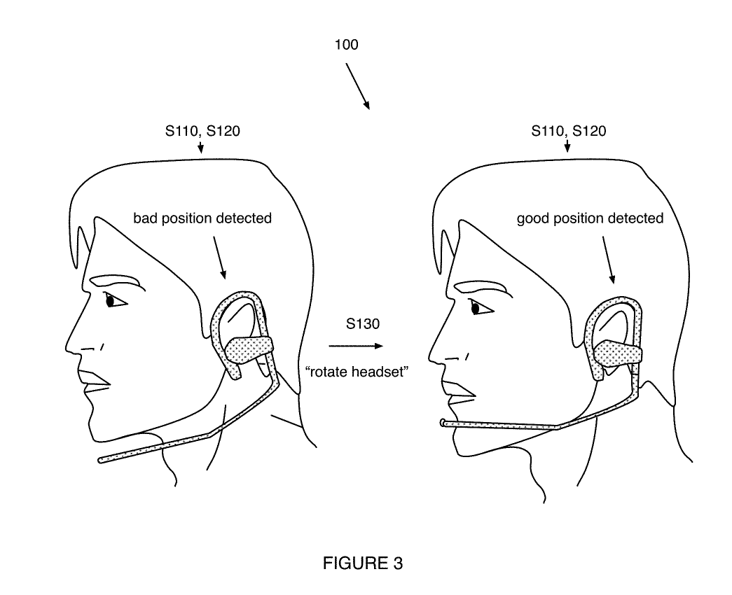

Block S130 can also function to provide specific guidance for sensor placement adjustments to improve bioelectrical contact quality, based on the output of Block S120 (e.g., the contact quality metric, based on the direct electrical measurement). For example, Block S130 can include instructing the user to insert one or more sensors more deeply into an ear canal region, based on a signal feature extracted in accordance with Block S120 having an RMS value below a threshold value. In another example, as shown in FIG. 3, Block S130 can include instructing the user to rotate the biomonitoring neuroheadset until a good position is detected. However, Block S130 can additionally or alternatively include providing any suitable form of qualitative and/or direct notification related to monitored contact characteristics.

Block S130 can include notifying a user of signal quality (e.g., contact quality, signal and contact quality, just signal quality, etc.). Notifying a user regarding signal quality preferably includes notifying a user in real-time regarding the signal quality for one or more sensors of the biomonitoring neuroheadset. Notifying a user can include providing a visual notification (e.g., a notification presented at a user interface of the biomonitoring neuroheadset, a push notification at a smartphone of a user as shown in FIG. 2, etc.), an auditory notification (e.g., sounds emitted through a speaker of the biomonitoring neuroheadset as shown in FIG. 2, etc.), a haptic notification (e.g., a vibration of the biomonitoring neuroheadset), and/or any other suitable type of notification. However, notifying a user of signal quality can be performed in any suitable manner.

Block S130 can additionally or alternatively include processing the signal data from one or more sensors based on a detected and/or calculated signal quality. In some variations for instance, sections of signal data having excessive noise can be any or all of: ignored (e.g., cut from the overall signal data stream), subjected to more intense processing (e.g., intense filtering), down-weighted when developing new detections (e.g., models, algorithms, etc.) or applying existing ones (e.g., by providing a confidence metric which indicates the expected accuracy of each segment or data), or used in any other suitable way.

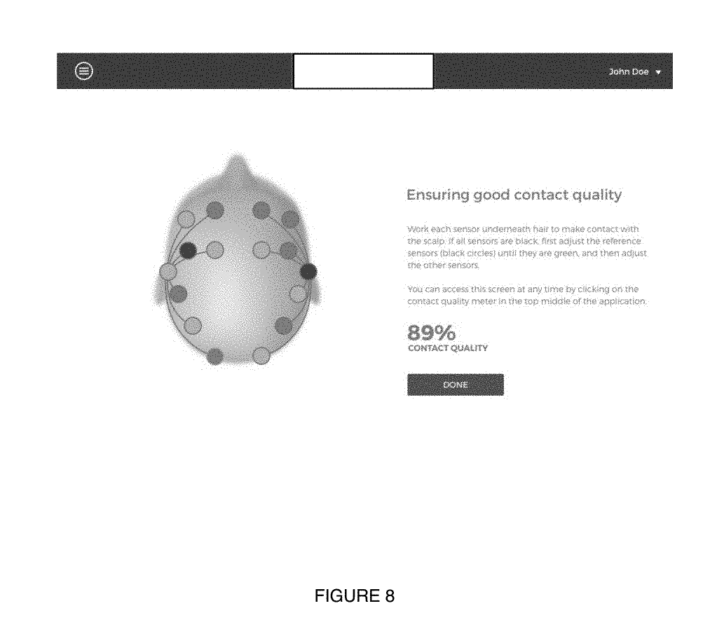

In one variation, one or more notifications can be provided through a visual indicator (e.g., graphic), such as through a display of a user device (e.g., mobile phone). The visual indicator can include any or all of: a spectrum bar, gradient, a set of colored dots corresponding to individual sensors and indicating a signal quality associated with each sensor, or any other suitable visual indicator. In a specific example, a visual representation of the sensors in the system is provided at a display of a user device (e.g., through a client application executing on the user device), wherein the visual representation of each of the sensors is assigned a particular color corresponding to quality of signal and/or contact (e.g., as shown in FIG. 8). An indication that a particular sensor is shown in red, for instance, can notify the user to try moving or otherwise manipulating the sensor to try to achieve better contact with the user's skin. Additionally or alternatively, one or more notifications can be provided audibly to a user (e.g., through a speaker of the system, through a speaker of a user device, etc.), through a written notification (e.g., text message), through a haptic stimulus (e.g., through a vibration motor associated with an electrode), or through any other suitable means.

In a second variation, a notification provided to the user is determined, at least in part, by sensor (e.g., electrode) type. In the event that the system includes dry sensors, for instance, the notification can include instructing the user to adjust one or more sensors, whereas in the event that the system includes wet sensors (e.g., saturated with a conductive gel), the notification can include instructing the user to apply more conductive fluid to the sensors. Additionally or alternatively, the instruction can depend on the category of sensor (e.g., reference electrode, common mode sensor, standard EEG electrode, etc.). In one example, if a single EEG electrode has poor signal quality, no notification is sent (e.g., because of redundancy in the EEG electrodes), whereas if a reference sensor has poor signal quality, a notification is sent to the user to adjust the reference electrode.

In a third variation, a notification is provided audibly to a user. In an example of the system having speakers (e.g., for music playback), notifying the user can include providing audio signals (e.g., “move the sensor closest to the right ear down by one inch”) to the user instructing her to move or otherwise manipulate sensors experiencing poor contact.

The method and/or system of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a patient computer or mobile device, or any suitable combination thereof. Other systems and methods of the embodiments can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, though any suitable dedicated hardware device can (alternatively or additionally) execute the instructions.

### 4. Variations

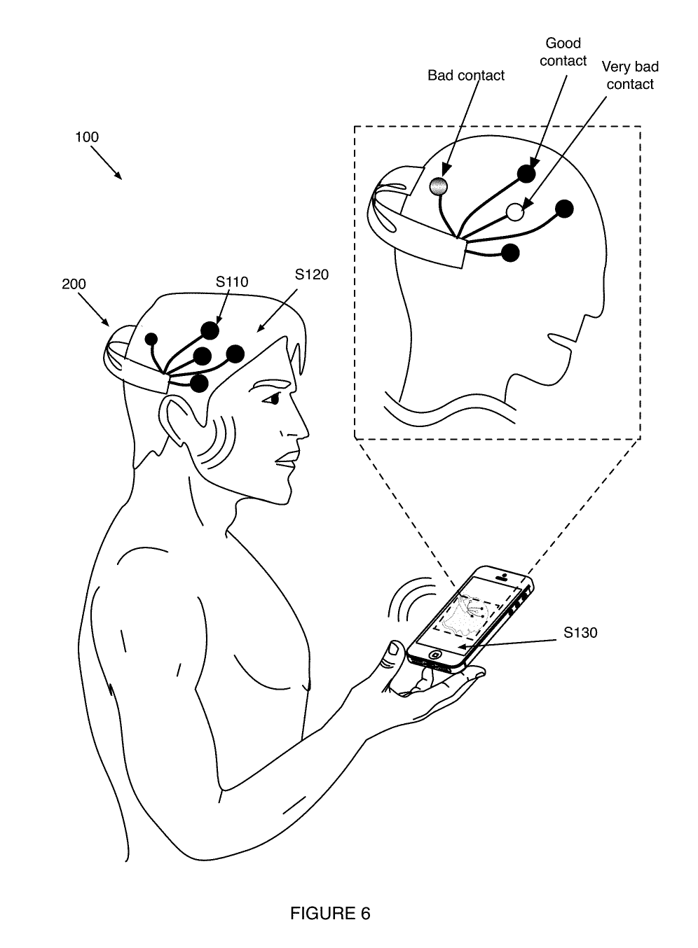

In one variation of the method (e.g., as shown in FIG. 6), the method 200 includes: establishing bioelectrical contact between the user and a set of EEG electrodes, wherein the set of EEG electrodes are placed in contact with the user through a head apparatus; determining a contact quality of each the set of electrodes through a direct electrical measurement process (e.g., transmitting a set of square wave signals to each of the set of electrodes); determining a full signal quality of each of the set of electrodes, wherein determining the full signal quality includes determining a set of features (e.g., frequency features, energy features, and gradient features) associated with an EEG signal from one or more of the set of electrodes and comparing the set of features with a signal quality model (e.g., probabilistic model, Gaussian model, etc.) to determine a signal quality metric (e.g., probability of good signal quality); indicating through a graphic representation of the set of electrodes at a display of a user device which electrodes are in proper contact with the user; and suggesting that the user adjust the electrodes which are not in proper contact with the user. Additionally or alternatively, the method can include any other suitable processes.

In a second variation of the method (e.g., as shown in FIG. 2), the method 200 includes: establishing bioelectrical contact between the user and a pair of EEG electrodes, wherein each of the pair of EEG electrodes is placed within an ear canal of the user; determining a contact quality of each the pair of electrodes through a direct electrical measurement process (e.g., transmitting a set of square wave signals to each of the set of electrodes); determining a full signal quality of each of the pair of electrodes, wherein determining the full signal quality includes determining a set of features (e.g., frequency features, energy features, and gradient features) associated with an EEG signal from each of the pair of electrodes and comparing the set of features with a signal quality model (e.g., probabilistic model, Gaussian model, etc.) to determine a signal quality metric (e.g., probability of good signal quality); indicating through a pair of speakers arranged proximal to the pair of electrodes that one or more of the electrodes is not properly contacting the user; and suggesting that the user adjust (e.g., rotate, place further within the ear canal, etc.) the electrodes which are not in proper contact with the user. Additionally or alternatively, the method can include any other suitable processes.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the embodiments of the invention without departing from the scope of this invention as defined in the following claims.

We claim:

1. A method for determining a signal quality metric of a set of electroencephalography (EEG) signals received at a set of sensors, the set of sensors configured to be arranged proximal to a head region of a user, the method comprising:
based on a set of EEG signals, determining a model associated with a signal quality of an EEG signal;
for each of the set of sensors of the user, determining a value for each of a set of features associated with an EEG signal of the sensor, wherein the set of features comprises at least a first, second, and third feature, wherein:
the first feature comprises a power parameter associated with a predetermined frequency range of the EEG signal;
the second feature comprises an overall power parameter associated with the EEG signal; and
the third feature comprises a gradient parameter associated with the EEG signal;
determining a signal quality metric based on the probabilistic model and the values of the set of features; and
providing a notification to the user, wherein the notification comprises an instruction to the user to adjust at least one of the set of sensors, and wherein the notification is determined based on the signal quality metric.

2. The method of claim 1, further comprising determining a contact quality metric, wherein determining the contact quality metric comprises delivering a predetermined signal to each of the set of electrodes and measuring a response at each of the set of electrodes.

3. The method of claim 2, wherein the predetermined signal comprises a square wave potential injected into a driven right leg signal.

4. The method of claim 2, wherein determining the value associated with each of the set of features is performed in response to determining that at least one of the set of responses differs from the predetermined signal by an amount greater than a predetermined threshold.

5. The method of claim 1, wherein determining the value for each of the set of features for each of the set of sensors comprises a sliding window process.

6. The method of claim 5, wherein the values of each of the set of features are determined between every eighth of a second and every ten seconds.

7. The method of claim 6, wherein each of a set of windows used in the sliding window process has a length between 1 and 4 seconds.

8. The method of claim 1, wherein the model comprises a probabilistic model determined based on signal data received from an aggregated set of users, wherein the signal data is determined by an expert to have a signal quality above a predetermined threshold.

9. The method of claim 8, wherein signal data having a signal quality below the predetermined threshold are excluded from use in determining the probabilistic model.

10. The method of claim 1, wherein the predetermined frequency band of the signal comprises a frequency associated with mains noise of an environment of the user.

11. The method of claim 10, wherein the predetermined frequency band includes a frequency between 50 and 60 Hertz.

12. The method of claim 1, wherein the overall power parameter is a root mean square (RMS) power.

13. The method of claim 1, wherein the gradient parameter comprises at least one of a sum of an absolute gradient at each of a set of time points of the EEG signal and a gradient measure in frequency space which identifies spikes from mains noise and aliased harmonics.

14. A method for determining a signal quality metric of a set of electroencephalography (EEG) signals received at a set of sensors, the set of sensors configured to be arranged proximal to a head region of a user, the method comprising:
for each of the set of sensors of the user, determining a contact quality associated with the sensor, wherein determining the contact quality comprises delivering a signal to each of the set of sensors and measuring a response at each of the set of sensors;
for each of the set of sensors of the user, determining a value for each of a set of features associated with an EEG signal of the sensor, wherein the set of features comprises a power parameter and a gradient parameter;
determining a signal quality metric based on the contact quality, a model and the values of the set of features;
providing a notification to the user at a user device associated with the user, wherein the notification comprises an instruction to the user to adjust at least one of the set of sensors, and wherein the notification is determined based on the signal quality metric.

15. The method of claim 14, wherein the set of features comprises at least a first, second, and third feature, wherein:
the first feature comprises a power parameter associated with a predetermined frequency range of the EEG signal;
the second feature comprises an overall power parameter associated with the EEG signal; and
the third feature comprises a gradient parameter associated with the EEG signal.

16. The method of claim 14, further comprising determining the model, wherein the model comprises a probabilistic model, based on a set of EEG signals received from an aggregated set of users, wherein each of the set of EEG signals is determined by an expert to have a signal quality above a predetermined threshold.

17. The method of claim 14, wherein the signal comprises a square wave potential injected into a driven right leg signal.

18. The method of claim 14, wherein the notification further comprises a graphic provided at a display of the user device, the graphic comprising a virtual representation of each of the set of sensors.

19. The method of claim 18, wherein a color of the virtual representation of each of the set of sensors is determined based on the signal quality metric associated with the sensor.

20. The method of claim 14, wherein determining the value for each of the set of features for each of the set of sensors comprises a sliding window process, wherein in the sliding window process:
the values of each of the set of features are determined between every eighth of a second and every second; and
each of a set of windows used in the sliding window process has a length between 0.5 and 10 seconds.

21. The method of claim 1, further comprising collecting movement data associated with the user from a motion sensor, wherein the notification is further determined based on the movement data.
